(19) United States
(12) Patent Application Publication
(10) Pub. No.: US 2018/0299442 A1
(43) Pub. Date: Oct. 18, 2018

(54) ENRICHMENT AND IDENTIFICATION OF FETAL MATERIAL

(71) Applicant: Jennifer C. Chow, Seattle, WA (US)

(72) Inventor: Jennifer C. Chow, Seattle, WA (US)

(21) Appl. No.: 15/949,733

(22) Filed: Apr. 10, 2018

Related U.S. Application Data
(60) Provisional application No. 62/485,470, filed on Apr. 14, 2017.

(51) Int. Cl. G01N 33/569; G01N 1/40; C12Q 1/6883

(52) U.S. Cl. CPC G01N 33/56966; G01N 1/4077; G01N 2333/71; G01N 2333/705; G01N 2333/988; C12Q 1/6883

(57) ABSTRACT

This disclosure is directed to a kit and method for retrieving a target material, such as fetal material, from a sample, such as a maternal sample or fraction thereof. An enrichment agent can be added to a vessel that contains the sample for positive selection, or, in other words, to select or aid in selecting the target material from amongst the remainder of the sample. The enrichment agent can be, for example, immunomagnetic beads, buoyant beads, high-density beads, chemicals to change the density of the target material, or the like.

### CROSS REFERENCE TO RELATED APPLICATION

This application claims the benefit of Provisional Application No. 62/485,470, filed Apr. 14, 2017.

### TECHNICAL FIELD

This disclosure relates generally to sample enrichment and, in particular, to retrieving a target material or target analyte from a sample.

### BACKGROUND

Samples, such as biological samples, often include materials of interests that are difficult to detect, extract and isolate for analysis. Currently, practitioners, researchers, and those working with biological samples, for example, try to separate, isolate, and extract certain components of the biological sample for examination. As a result, practitioners, researchers, and those working with suspensions also continue to seek systems and methods for enrichment and analysis of biological samples for the presence of absence of particular materials of interest.

### DETAILED DESCRIPTION

This disclosure is directed to a kit and method for retrieving a target material, such as fetal material, from a sample, such as a maternal sample or fraction thereof. An enrichment agent can be added to a vessel that contains the sample for positive selection, or, in other words, to select or aid in selecting the target material from amongst the remainder of the sample. The enrichment agent can be, for example, immunomagnetic beads, buoyant beads, high-density beads, chemicals to change the density of the target material, or the like.

In the following descriptions, the term “light” is used to describe various uses and aspects of multiplexing and imaging. The term light is not intended to be limited to describing electromagnetic radiation in the visible portion of the electromagnetic spectrum, but is also intended to describe radiation in the ultraviolet and infrared portions of the electromagnetic spectrum.

In the following descriptions, the term “sample” is used to describe a biological fluid, a biological semi-solid, a biological solid (which can remain solid, such as tissue, or can be liquefied in any appropriate manner), a suspension, a portion of the suspension, a component of the suspension, or the like. For example, for anticoagulated whole blood, the sample is the anticoagulated whole blood (i.e. a suspension), the buffy coat (i.e. a portion of the suspension), or a circulating tumor cell (i.e. a component of the suspension). For the sake of convenience, the sample referenced is whole blood, though it should be understood that the method and system described and discussed herein is used with any appropriate sample, such as urine, blood, bone marrow, buffy coat, cystic fluid, ascites fluid, stool, semen, cerebrospinal fluid, nipple aspirate fluid, saliva, amniotic fluid, mucus membrane secretions, aqueous humor, vitreous humor, vomit, vaginal fluid, and any other physiological fluid or semi-solid. For example, the sample is a tissue sample or a material from adipose tissue, an adrenal gland, bone marrow, a breast, a caudate, a cerebellum, a cerebral cortex, a cervix, a uterus, a colon, an endometrium, an esophagus, a fallopian tube, a heart muscle, a hippocampus, a hypothalamus, a kidney, a liver, a lung, a lymph node, an ovary, a pancreas, a pituitary gland, a prostate, a salivary gland, a skeletal muscle, skin, a small intestine, a large intestine, a spleen, a stomach, a testicle, a thyroid gland, or a bladder.

In the following descriptions, the term “maternal sample” is used to describe any sample collected, withdrawn, isolated, or the like from a pregnant female that is not collected directly from the fetus or any biological structure associated directly with or aiding in fetal growth or development (e.g., placenta, amniotic fluid, and umbilical cord). For clarification purposes, the maternal sample can include material shed, sloughed, and/or disbursed by the fetus or associated biological structure (e.g., fetal cells, fetal trophoblasts fetal DNA, fetal RNA, etc.).

In the following descriptions, the terms “target analyte” or “target material” are used to describe a biological material of interest. It should also be understood that the target analyte can be a fraction of a sample, such as buffy coat, a cell, such as ova, fetal material (such as trophoblasts, nucleated red blood cells, fetal red blood cells, fetal white blood cells, fetal DNA, fetal RNA, or the like), a circulating tumor cell (“CTC”), a circulating endothelial cell, an immune cell (i.e. naïve or memory B cells or naïve or memory T cells), a mesenchymal cell, a stem cell, a vesicle, such as an exosome, a liposome, a protein, a nucleic acid, a biological molecule, a naturally occurring or artificially prepared microscopic unit having an enclosed membrane, parasites (e.g. spirochetes, such as Borrelia burgdorferi which cause Lyme disease; malaria-inducing agents), microorganisms, viruses, or inflammatory cells. For example, the target analyte is a tumor cell from adipose tissue, an adrenal gland, bone marrow, a breast, a caudate, a cerebellum, a cerebral cortex, a cervix, a uterus, a colon, an endometrium, an esophagus, a fallopian tube, a heart muscle, a hippocampus, a hypothalamus, a kidney, a liver, a lung, a lymph node, an ovary, a pancreas, a pituitary gland, a prostate, a salivary gland, a skeletal muscle, skin, a small intestine, a large intestine, a spleen, a stomach, a testicle, a thyroid gland, or a bladder.

In the following descriptions, the term “non-target analyte” is used to describe a biological material which is not a target analyte.

In the following descriptions, the term “biomarker” is used to describe a substance that is present on or within the target analyte or target material (i.e. intracellular or extracellular the target analyte; internalized, such as through phagocytosis, within the target analyte; or the like). Biomarkers include, but are not limited to, peptides, proteins, subunits, domains, motifs, epitopes, isoforms, DNA, RNA, or the like. The biomarker can be a target molecule for drug delivery.

In the following descriptions, the term “affinity molecule” is used to describe any molecule that is capable of binding or interacting with a biomarker. The interaction or binding can be covalent or non-covalent. The affinity molecule includes, but is not limited to, an antibody, a hapten, a protein, an aptamer, an oligonucleotide, a polynucleotide, or any appropriate molecule for interacting with or binding to the biomarker.

In the following descriptions, the term “detection moiety” is used to describe a compound or substance which provides a signal for detection, thereby indicating the presence of another compound or substance, an analyte, or the like within a sample or specimen. The detection moiety can be fluorescent, such as a fluorescent probe, or chromogenic, such as a chromogenic dye.

In the following descriptions, the term “channel” is used to describe a color or color range based on the signal provided by one or more detection moieties. The color or color range is obtained based on the filters chosen and/or the wavelength of the signal(s). For example, a channel may be violet, blue, green, yellow, orange, red, dark red, or the like. Furthermore, when a plurality of channels are used, each channel has a specific color or color range. For example, a first channel may be green and a second channel may be orange. It should be noted that although two or more detection moieties may provide signals having different wavelengths, the signals can be in the same channel based on the filter set used. For example, a first detection moiety provides signal having a wavelength of 488 and a second detection moiety provides a signal having a wavelength of 500. Even though the wavelengths are not identical, the filter set in one of the channels passes wavelengths of both 488 nm and 500 nm, which permits both to be imaged at the same time, thereby producing a single image including the 488 and 500 emissions.

In the following descriptions, the terms “stain” or “label,” which are used interchangeably, are used to describe an affinity molecule bound to or interacted with a detection moiety. The binding or interaction can be direct or indirect. Direct binding or interaction includes covalent or non-covalent interactions between the biomarker and the detection moiety. Indirect binding or interaction includes the use of at least first and second complementary molecules which form binding pairs. The first and second complementary molecules are, in combination, binding pairs which binds or interacts in at least one of the following manners: hydrophobic interactions, ionic interactions, hydrogen bonding interactions, non-covalent interactions, covalent interactions, affinity interactions, or the like. The binding pairs include, but are not limited to, immune-type binding-pairs, such as, antigen-antibody, antigen-antibody fragment, hapten-anti-hapten, or primary antibody-secondary antibody; nonimmune-type binding-pairs, such as biotin-avidin, biotin-streptavidin, folic acid-folate binding protein, hormone-hormone receptor, lectin-specific carbohydrate, enzyme-enzyme, enzyme-substrate, enzyme-substrate analog, enzyme-pseudo-substrate (substrate analogs that cannot be catalyzed by the enzymatic activity), enzyme-cofactor, enzyme-modulator, enzyme-inhibitor, or vitamin B12-intrinsic factor. Other suitable examples of binding pairs include complementary nucleic acid fragments (including complementary nucleotides, oligonucleotides, or polynucleotides); Protein A-antibody; Protein G-antibody; nucleic acid-nucleic acid binding protein; polymeric linkers (e.g., polyethylene glycol); or polynucleotide-polynucleotide binding protein. The binding pairs can be included within or used as amplification techniques. Amplification techniques are also implemented to increase the number of detection moieties bound to or interacted with the biomarker to increase a signal.

Furthermore, “a plurality of stains” can be used to describe two or more stains in which the affinity molecules and/or the detection moieties are different. For example, anti-CK-Alexa 647 is different than anti-EPCAM-Alexa 647. As another example, anti-CK-Alexa 647 is different than anti-CK-Alexa 488.

In the following descriptions, the terms “permeabilize” or “permeabilization” are used to describe the dissolution or removal of a portion of a plasma membrane of a target or non-target analyte by chemical or other means, such that at least an IgG antibody is capable of crossing the plasma membrane.

In the following descriptions, the term “conjugate” is used to describe a first chemical, molecule, moiety, particle, or the like bound to or interacted with a second chemical, molecule, moiety, particle, or the like. The binding or interaction can be direct or indirect. Direct binding or interaction includes covalent or non-covalent interactions between the biomarker and the detection moiety. Indirect binding or interaction includes the use of at least first and second complementary molecules which form binding pairs. The first and second complementary molecules are, in combination, binding pairs which binds or interacts in at least one of the following manners: hydrophobic interactions, ionic interactions, hydrogen bonding interactions, non-covalent interactions, covalent interactions, affinity interactions, or the like. The binding pairs include, but are not limited to, immune-type binding-pairs, such as, antigen-antibody, antigen-antibody fragment, hapten-anti-hapten, or primary antibody-secondary antibody; nonimmune-type binding-pairs, such as biotin-avidin (as shown in FIG. 1B), biotin-streptavidin, folic acid-folate binding protein, hormone-hormone receptor, lectin-specific carbohydrate, enzyme-enzyme, enzyme-substrate, enzyme-substrate analog, enzyme-pseudo-substrate (substrate analogs that cannot be catalyzed by the enzymatic activity), enzyme-cofactor, enzyme-modulator, enzyme-inhibitor, or vitamin B12-intrinsic factor. Other suitable examples of binding pairs include complementary nucleic acid fragments (including complementary nucleotides, oligonucleotides, or polynucleotides); Protein A-antibody; Protein G-antibody; nucleic acid-nucleic acid binding protein; polymeric linkers (e.g., polyethylene glycol); or polynucleotide-polynucleotide binding protein. In the following description, the term “fetal material” is used to describe any biological material that is present in a pregnant female as a result of the pregnancy.

Furthermore, “a plurality of conjugates” can be used to describe two or more conjugates in which the first chemical, molecule, moiety, particle, or the like and/or the second chemical, molecule, moiety, particle, or the like are different. For example, anti-EPCAM-immunomagnetic bead is different than anti-LVRN-immunomagnetic bead. As another example, anti-EPCAM-immunomagnetic bead is different than anti-EPCAM-glass bead.

The term “fetal material” can be used to describe material resulting from the pregnancy of a female, and which can include, but is not limited to, trophoblasts, nucleated red blood cells, fetal red blood cells, fetal white blood cells, fetal DNA, or fetal RNA.

An example method for detecting fetal material is discussed. For the sake of convenience, the methods are described wherein the sample is buffy coat, such as buffy coat having been enriched from maternal blood, and the target material or target analyte is a fetal trophoblast. But the methods described below are not intended to be so limited in their scope of application. In one embodiment, the sample is a fraction of a suspension, such that the sample is obtained through enrichment, including positive and/or negative enrichment and/or density-based enrichment. The enriched fraction is suspected of including at least one target analyte. The sample is enriched by any appropriate enrichment process including, but not limited to, sequential density fractionation, magnetic-activated cell sorting, fluorescence-activated cell sorting, differential lysis, depletion filters, microfluidic device separation, or the like. In one embodiment, the sample is the suspension. Suitable devices, systems, and/or methods of sample collection and/or processing may include those described in one or more of the following U.S. patents and published applications, each of which is hereby incorporated by reference in its entirety: U.S. Pat. Nos. 7,074,577; 7,220,593; 7,329,534; 7,358,095; 7,629,176; 7,915,029; 7,919,049; 8,012,742; 9,039,999; 9,217,697; 9,492,819; 9,513,291; 9,533,303; 9,539,570; 9,541,481; 9,625,360; 2014/0161688; 2017/0014819; 2017/0059552; 2017/0074759. Suitable devices, systems, and/or methods for target analyte retrieval, isolation, or picking may include those described in one or more of the following U.S. patents and published applications, each of which is hereby incorporated by reference in its entirety: U.S. Pat. Nos. 9,222,953; 9,440,234; 9,519,002; 9,810,605; 2017/0219463; 2017/0276575.

In one embodiment, prior to enrichment, a depletion agent can be added to maternal blood after collection from a patient or subject to remove a fraction of the maternal blood or to change the density of at least a fraction of the maternal blood relative to the density of the buffy coat. For example, the depletion agent can be used to move platelets away from the buffy coat, such as by changing the density of the platelets to be greater than at least the buffy coat, though the density of the platelets can be made to be greater than or equal to the red blood cells. In one embodiment, the depletion agent is not added to the maternal blood.

Examples of suitable depletion agents include solutions such as, a solution of colloidal silica particles coated with polyvinylpyrrolidone (e.g. Percoll), polysaccharide solution (e.g. Ficoll), iodixanol (e.g. OptiPrep), a complex, branch glucan (e.g. Dextran), cesium chloride, sucrose, sugar-based solutions, polymer solutions, multi-phase polymer solutions, tetrameric antibody complexes (e.g. RosetteSep) or the like; or particles, such as beads (composed of at least one of a metal, silica, glass, a polymer, or the like), nanoparticles, metal-based compounds, metal complexes, lipids, sugars, or the like. The particles can be approximately 0.1-5.0 μm in size. In one embodiment, the depletion agent is directly conjugated to an affinity molecule to bind to a biomarker of the non-target material. In one embodiment, the depletion agent is indirectly conjugated to an affinity molecule to bind to a biomarker of the non-target material. In one embodiment, the depletion agent is not conjugated, directly or indirectly, to an affinity molecule.

In one embodiment, after collecting the buffy coat from the maternal blood, an enrichment agent can be added to the buffy coat to select or aid in selecting a fetal trophoblast from the collected constituent component. The enrichment agent can alter a physical characteristic of the target material (i.e. density, ability to respond to a magnetic gradient, or the like). The particles or beads can be approximately 0.1-5.0 μm in size. Examples of suitable enrichment agents include, without limitation, solutions such as, a solution of colloidal silica particles coated with polyvinylpyrrolidone (e.g. Percoll), polysaccharide solution (e.g. Ficoll), iodixanol (e.g. OptiPrep), a complex, branch glucan (e.g. Dextran), cesium chloride, sucrose, sugar-based solutions, polymer solutions, multi-phase polymer solutions, tetrameric antibody complexes (e.g. RosetteSep) or the like; or particles, such as beads (composed of at least one of a metal, silica, glass, a polymer, or the like), nanoparticles, metal-based compounds, metal complexes, lipids, sugars, immunomagnetic beads, buoyant beads, high-density beads, chemicals to change the density of the target material, or the like. In one embodiment, one type of enrichment agent can be used.

The enrichment agent can be directly or indirectly conjugated to an affinity molecule. In one embodiment, one affinity molecule directed to one biomarker can be used, whereby the enrichment agent is conjugated to the affinity molecule. The affinity molecules include, without limitation, HLA-G, EGFR, HER-2, HER-3, HER-4, CD105, CD144, CD147, EPCAM, PSMA, PSA, CD271, MUC1, LVRN, E-cadherin, N-cadherin, CD71, and, CD146.

In one embodiment, a cocktail having two or more affinity molecules directed to two or more biomarkers can be used, whereby two or more of the same type of enrichment agent is conjugated to two or more biomarkers. For example, magnetic beads can be conjugated to EPCAM, LVRN, EGFR, and HER-2.

In one embodiment, a plurality of enrichment agents can be used, whereby each type of enrichment agent is conjugated to a different affinity molecule. For example, immunomagnetic beads can be conjugated to EPCAM and buoyant glass beads can be conjugated to EGFR.

In one embodiment, a cocktail having three or more affinity molecules directed to three of more biomarkers can be used, whereby two of the affinity molecules are conjugated to a first enrichment agent and the at least one other affinity molecule is conjugated to a second enrichment agent. For example, immunomagnetic beads can be conjugated to EPCAM and HER-2, and buoyant glass beads can be conjugated to EGFR.

In one embodiment, the affinity molecule-enrichment agent conjugate can be pre-conjugated and added to the sample. In one embodiment, the affinity molecule and the enrichment agent are added separately to the sample and form the conjugate, such as via binding pairs, within the sample.

In one embodiment, such as when immunomagnetic beads are used, a magnet can be placed proximal to the vessel containing the buffy coat. The fetal trophoblast, via the bound immunomagnetic beads, is attracted to a wall or a surface of the vessel. At least a portion of non-target material of the buffy coat is not attracted to the wall or the surface of the vessel, since that material is not bound to immunomagnetic beads. The non-target material can be removed from the vessel, such as by pipetting or pouring off.

In one embodiment, the enrichment agent can be cleaved, separated, removed, or detached from the affinity molecule, such as by a chemical, light, heat, enzyme, or any appropriate method. This can occur at any appropriate point in the process, such that no subsequent step uses a device (e.g., a magnet) that can interact with the enrichment agent for further or additional processing. For example, the enrichment agent can be separated from the affinity molecule after placing the tube containing the sample near the magnet and removing the non-attracted material from the tube, and before labeling or staining.

In one embodiment, a reagent can be added to the vessel, such as to permeabilize the fetal trophoblast.

In one embodiment, the fetal trophoblast can be stained or labeled after collection of the buffy coat from the maternal blood. Staining or labeling can performed in the same vessel in which the enrichment steps occurs or in or on a different vessel, such as another tube or on a slide. In one embodiment, at least one stain is added before the enriching step. In one embodiment, at least one stain is added after the enriching step. In one embodiment, at least one stain and the enrichment agent are added simultaneously. In one embodiment, at least one stain can be added before the enriching step and at least one stain can be added after the enriching step. In one embodiment, the stain, regardless of when it is added, can include or incorporate amplification. In one embodiment, the stain, regardless of when it is added, does not include or incorporate amplification. When a plurality of stains is used, one stain can include or incorporate amplification, some of the stains can include or incorporate amplification, or all of the stains can include or incorporate amplification. The amplification techniques include, but are not limited to, haptens and anti-haptens (such as DNP and anti-DNP; DIG and anti-DIG; FITC and anti-FITC; HQ and anti-HQ; and biotin), primary and secondary antibodies, horseradish peroxidase (HRP) and tyramide, HRP or alkaline phosphatase and 3,3′ diaminobenzidine (DAB) or 3-amino-9-ethylcarbazole (AEC), and complementary molecules (such as biotin and an avidin (e.g., streptavidin or neutravidin)).

The detection moiety can be fluorescent, such as a fluorescent probe, or chromogenic, such as a chromogenic dye. The chromogenic dye, which can be used with various enzyme labels (e.g. horseradish peroxidase and alkaline phosphate), includes, but is not limited to, 3,3′-Diaminobenzidine (DAB), 3-Amino-9-Ethylcarbazole (AEC), 4-Chloro-l-Naphtol (CN), P-Phenylenediamine Dihydrochloride/pyrocatechol (Hanker-Yates reagent), Fast Red TR, New Fuchsin, Fast Blue BB, or the like. The fluorescent probe can be a reactive dye, an organic dye, a fluorescent protein, a quantum dot, non-protein organic molecules, a nanoparticle (e.g., nanodiamond), or the like. Fluorescent probes include, but are not limited to 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; AB Q; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); AutoFluorescent Protein; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9(Bisaminophenyloxadiazole); BCECF (high pH); BCECF (low pH); Berberine Sulphate; Beta Lactamase; BFP blue shifted GFP (Y66H; Blue Fluorescent Protein); BFP/GFP FRET; Bimane; Bisbenzamide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Brilliant Violet 421; Brilliant Violet 510; Brilliant Violet 605; Brilliant Violet 650; Brilliant Violet 711; Brilliant Violet 786; BTC; BTC-5N; Calcein; Calcein Blue; Calcium Crimson™; Calcium Green; Calcium Green-1; Calcium Green-2; Calcium Green-5N; Calcium Green-C18; Calcium Orange; Calcofluor White; Carboxy-X-hodamine (5-ROX); Cascade Blue™; Cascade Yellow; Catecholamine; CCF2 (GeneBlazer); CFDA; CFP (Cyan Fluorescent Protein); CF405S; CF488A; CF 488; CF 543; CF 647; CF 750; CF 760; CF 780; FP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); CyQuant Cell Proliferation Assay; Dabcyl; Dansyl; Dansyl Amine; Dansyl Cadaverine; Dansyl Chloride; Dansyl DHPE; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS; DiA (4-Di-16-ASP); Dichlorodihydrofluorescein Diacetate (DCFH); DiD-Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DM-NERF (high pH); DNP; Dopamine; DsRed; DTAF; DY-630-NHS; DY-635-NHS; EBFP (Enhanced Blue Fluorescent Protein); ECFP (Enhanced Cyan Fluorescent Protein); EGFP (Enhanced Green Fluorescent Protein); ELF 97; Eosin; ER-Tracker™ Green; ER-Tracker™ Red; ER-Tracker™ Blue-White DPX; Erythrosin; Erythrosin ITC; Ethidium Bromide; Ethidium homodimer-1 (EthD-1); Euchrysin; EukoLight; Europium (III) chloride; EYFP (Enhanced Yellow Fluorescent Protein); Fast Blue; FDA; FIF (Formaldehyde Induced Fluorescence); FITC; FITC Antibody; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2, high calcium; Fura-2, low calcium; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green; Mitotracker Orange; Mitotracker Red; Mitramycin; Monobromobimane; Monobromobimane (mBBr-GSH); Monochlorobimane; MPS (Methyl Green Pyronine Stilbene); mStrawberry; NBD; NBD Amine; Nile Red; Nitrobenzoxadidole; Noradrenaline; Nuclear Fast Red; Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B; Phycoerythrin R; PKH26 (Sigma); PKH67; PMIA; Pontochrome Blue Black; POPO-1; POPO-3; PO-PRO-1; PO-PRO-3; Primuline; Procion Yellow; Propidium Iodid (PI); Pyrene; Pyronine; Pyronine B; Pyrozal Brilliant Flavin 7GF; QD500; QD525; QD625; QD800; QSY 7; Quinacrine Mustard; Red 613 (PE-TexasRed); Resorufin; RFP; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin; rsGFP (red shifted GFP (S65T)); S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgGFP™ (super glow GFP; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; SYTOX Red; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TetramethylRodaminelsoThioCyanate; True Blue; TruRed; Tubulin Tracker™ Green; Ultralite; Uranine B; Uvitex SFC; wt GFP (wild type GFP); WW 781; X-Rhodamine; XRITC; Xylene Orange; Y66F; Y66H; Y66W; Yellow GFP (Yellow shifted); Green Fluorescent Protein; YFP (Yellow Fluorescent Protein); YO-PRO-1; YO-PRO-3; YOYO-1; YOYO-3; and, combinations and derivatives thereof.

It should be noted that a nuclear stain, such as Sytox, may be used in combination with a quantum dot. In one embodiment, the quantum dot has an emission peak less than or equal to 520 nm. In one embodiment, the quantum dot has an emission peak greater than or equal to 520 nm.

In one embodiment, after staining or labeling, such as when immunomagnetic beads are used, the magnet can be replaced proximal to the vessel containing the buffy coat. The fetal trophoblast, via the bound immunomagnetic beads, is re-attracted to a wall or a surface of the vessel. Any remaining non-target material of the buffy coat that is not attracted to the wall or the surface of the vessel, since that material is not bound to immunomagnetic beads. At least a portion of any remaining non-target material of the buffy or unbound material (e.g., unbound stain or reagent) can be removed from the vessel, such as by pipetting or pouring off.

In one embodiment, after staining or labeling, the buffy coat is imaged, whereby the buffy coat is illuminated with one or more wavelengths of excitation light from a light source, such as infrared, red, blue, green, and/or ultraviolet. The imaging can be done with a flow cytometer or a microscope, such as a fluorescent microscope, a scanner, or the like. Imaging can be done in brightfield and/or darkfield illumination, phase contrast, differential interference contrast, fluorescence, light sheet microscopy, super resolution microscopy, confocal microscopy, and Hoffman modulation contrast. The images formed can be overlaid when a plurality of detection moieties is used. Emission, reflection, diffraction, scatter, and combinations thereof are used in for detection/imaging. The images are analyzed to detect, enumerate, and locate the fetal trophoblast. Imaging is performed in a tube, on a microscope slide, or in any appropriate vessel or substrate for imaging.

The fetal trophoblast can be retrieved from the rest of the buffy coat. To retrieve the fetal trophoblast, the fetal trophoblast undergoes enrichment and/or isolation. The fetal trophoblast is isolated from rest of the buffy coat, whether with or without prior enrichment, by selecting the fetal trophoblast at a time with any appropriate device or system. Imaging the analysis platform, as discussed above, is performed to aid in isolation by providing location and characterization information for isolation purposes.

The fetal trophoblast can undergo post-processing analysis, such as sequencing, by using any appropriate method or technique, though more specifically extracellular and intracellular analysis including intracellular protein labeling; nucleic acid analysis, including, but not limited to, DNA arrays, expression arrays, protein arrays, and DNA hybridization arrays; or in situ hybridization (“ISH”—a tool for analyzing DNA and/or RNA, such as gene copy number changes); polymerase chain reaction (“PCR”); reverse transcription PCR. Sequencing is done on the entire genome, the transcriptome, or cDNA. Post-processing analysis can be performed to determine genetic abnormalities, including, without limitation, Down's Syndrome, trisomy 18, and neural tube defect.

### Biological Applications

The following is an example method for collecting fetal material, such as a fetal trophoblast, from maternal blood:

1. Collect buffy coat from maternal blood sample into a vessel
a. Vessel can be a tube, slide, or the like

2. Enrich for fetal trophoblast
a. Add cocktail of EpCAM-biotin, LVRN-biotin, EGFR-biotin, and HER2-biotin
b. Add immunomagnetic bead-streptavidin conjugates to the biotinylated cocktail

3. (Optional) Incubate

4. (Optional) Stain

5. Place the vessel in, near, underneath or on top of a magnet to draw the immunomagnetically-bound fetal trophoblast to the magnet
a. To the sidewall of a tube
b. To the bottom of a tube
c. To the surface of a slide

6. Withdraw magnet or vessel

7. Remove the non-immunomagnetically-bound material from the vessel

8. (Optional) Fix the fetal trophoblast

9. (Optional) Add at least one additional reagent to the vessel
a. Permeabilizing agent
b. Stains

10. (Optional) Place the vessel in, near, underneath or on top of a magnet to draw the immunomagnetically-bound fetal material to the magnet

11. (Optional) Withdraw magnet or vessel

12. (Optional) Remove the non-immunomagnetically-bound material from the vessel

13. (Optional) Store or image the fetal trophoblast
a. Move to another vessel or retain in same vessel

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific embodiments are presented by way of examples for purposes of illustration and description. They are not intended to be exhaustive of or to limit this disclosure to the precise forms described. Many modifications and variations are possible in view of the above teachings. The embodiments are shown and described in order to best explain the principles of this disclosure and practical applications, to thereby enable others skilled in the art to best utilize this disclosure and various embodiments with various modifications as are suited to the particular use contemplated. It is intended that the scope of this disclosure be defined by the following claims and their equivalents:

What is claimed is:

1. A method, comprising:
providing a maternal sample or a fraction thereof suspected of comprising fetal material; and
enriching the fetal material with at least two affinity molecule-enrichment agent conjugates,
wherein the at least two affinity molecules of the conjugates are directed to at least two of CD144, CD147, EPCAM, LVRN, EGFR, HLA-G, HER-2, HER-3, or HER-4, and
wherein the fetal material expresses at least one biomarker to which at least one of the at least two affinity molecules is directed.

2. The method of claim 1, further comprising
adding, to the maternal sample or fraction thereof, at least two affinity molecule-first complementary molecule conjugates; and
adding, to the maternal sample or fraction thereof, at least two second complementary molecule-enrichment agent conjugates,
wherein the first and second complementary molecules are binding pairs, thereby binding to or interacting with each other and forming the at least two affinity molecule-enrichment agent conjugates.

3. The method of claim 2, wherein the first complementary molecule is biotin and the second complementary molecule is an avidin.

4. The method of claim 1, wherein at least one of the at least two affinity molecule-enrichment agent conjugates, having been pre-conjugated, is added to the maternal sample or fraction thereof.

5. The method of claim 1, wherein the enrichment agent is immunomagnetic beads, buoyant beads, or high-density beads.

6. The method of claim 1, wherein the enrichment agent is immunomagnetic beads.

7. The method of claim 6, wherein the enriching step comprises
placing a magnet proximal to a vessel containing the maternal sample or fraction thereof,
wherein the fetal material, via the bound immunomagnetic beads, is attracted to a wall or a surface of the vessel.

8. The method of claim 7, further comprising removing material from the vessel that is not attracted to the wall or the surface of the vessel,
wherein the removing step is performed after the placing step.

9. The method of claim 8, further comprising labeling the fetal material with at least one stain.

10. The method of claim 9, further comprising imaging the at least one fetal material after labeling.

11. The method of claim 10, further comprising picking or isolating the fetal material.

12. The method of claim 11, further comprising sequencing the fetal material.

13. The method of claim 11, further comprising performing genetic testing on the fetal material.

14. The method of claim 13, wherein the genetic testing tests for at least one of Down's Syndrome, trisomy 18, and neural tube defect.

15. The method of claim 8, further comprising
replacing the magnet proximal to the vessel,
wherein the replacing step is performed after the removing step, and
wherein the replacing step is performed after having withdrawn the magnet prior to the replacing step.

16. The method of claim 15, further comprising labeling the fetal material with at least one stain before the replacing step.

17. The method of claim 16, further comprising labeling the fetal material with at least one other stain after the replacing step.

18. The method of claim 17, further comprising imaging the at least one fetal material after labeling with the at least one other stain.

19. The method of claim 18, further comprising picking or isolating the fetal material.

20. The method of claim 19, further comprising sequencing the fetal material.

21. The method of claim 19, further comprising performing genetic testing on the fetal material.

22. The method of claim 1, wherein the fetal material is a fetal cell, a fetal trophoblast, fetal DNA, fetal RNA, a fetal red blood cell, or a fetal white blood cell.

23. The method of claim 1, wherein the enriching step further comprises a CD105-enrichment agent conjugate, and wherein the fetal material expresses at least one of the biomarkers to which the at least three affinity molecules of the conjugates are directed.

24. The method of claim 1, further comprising cleaving, separating, removing, or detaching the enrichment agent from the affinity molecule after the enriching step.

25. The method of claim 24, wherein at least one of a chemical, light, heat, or enzyme is used to induce the cleaving, separating, removing, or detaching.

26. The method of claim 1, wherein enriching is performed with four affinity molecule-enrichment agent conjugates, wherein the four affinity molecules of the conjugates are directed to EPCAM, LVRN, EGFR, and HER-2, and wherein the fetal material expresses at least one biomarker to which at least one of the four affinity molecules is directed.

27. The method of claim 26, wherein the enriching step further comprises a CD105-enrichment agent conjugate, and wherein the fetal material expresses at least one of the biomarkers to which the five affinity molecules of the conjugates are directed.
